(19) United States
(12) Patent Application Publication
(10) Pub. No.: US 2009/0137383 A1
(43) Pub. Date: May 28, 2009

(54) CLASS OF ORGANIC COMPOUNDS CONTAINING HETEROATOM AND ITS APPLICATIONS IN PREPARING SINGLE-SITE ZIEGLER-NATTA CATALYST

(75) Inventors: Yong TANG, Shanghai (CN); Xiaohong Yang, Shanghai (CN); Bo Liu, Shanghai (CN); Xiuli Sun, Shanghai (CN); Zhi Ma, Shanghai (CN); Yuan Gao, Shanghai (CN); Cong Wang, Shanghai (CN)

Correspondence Address: PERKINS COIE LLP, POST OFFICE BOX 1208, SEATTLE, WA, 98111-1208, US

(73) Assignee: Shanghai Institute of Organic Chemistry, Chinese Academy of Sciences, Shanghai (CN)

(21) Appl. No.: 12/275,882

(22) Filed: Nov. 21, 2008

(30) Foreign Application Priority Data
May 22, 2006 (CN) 200610026766.2
May 21, 2007 (CN) PCT/CN2007/001648

(51) Int. Cl. B01J 29/04; C07C 221/00; C07F 9/28; B01J 21/16; B01J 31/02

(52) U.S. Cl. 502/62; 564/342; 564/15; 502/167; 502/84

(57) ABSTRACT

Organic compounds containing heteroatoms and their use in preparing Ziegler-Natta (Ziegler-Natta) catalyst with single activation center. The Ziegler-Natta olefin polymerization catalyst is preparing by adding organic or inorganic solid carrier or compound of them which is pre-activated by heating or pre-treated chemically, organic compound containing heteroatoms and metallic compound into magnesium compound/tetrahydrofuran solution. The Ziegler-Natta olefin polymerization catalyst prepared in the present invention is fluidizable powder and can prepare ethene homopolymer and copolymer of controllable construction with high catalytic activity, during homo-polymerization and combined polymerization with alpha-olefin of C₃˜C₁₈ under action of catalyst promoter such as alkyl aluminum, alkyl aluminoxane, and so on.

### RELATED APPLICATIONS

The present application is a continuation-in-part of PCT/CN2007/001648 filed on May 21, 2007 and published as WO 2007/134537 on Nov. 29, 2007, which in turn claims priority from Chinese Patent Application CN 200610026766.2 filed on May 22, 2006. The subject matter and contents of the PCT International application and priority application are incorporate herein by reference.

### FIELD OF THE INVENTION

The present invention provides a new class of organic compounds containing heteroatom, their syntheses, and applications as donor in preparing single-site Ziegler-Natta catalysts. Upon activation with alkyl aluminum, alkyl aluminoxane (MAO), or modified alkyl aluminoxane (MMAO), the single-site Ziegler-Natta catalysts can efficiently promote ethylene polymerization or ethylene/α-olefin copolymerization to provide high-performance polyolefin materials.

### BACKGROUND OF THE INVENTION

With the rapid development of polyolefin industry, much more extensive attention have been paid to the production of high-performance polyolefin materials. High-performance polyolefin materials can be prepared mainly in two ways: 1) by excellent single-site catalyst; 2) by advanced technology process. With single-site catalyst (homogeneous catalysts), the properties of polymer could be controlled well and so a variety of high-performance polyolefin materials are provided. However, metal complexes as the real active species of single-site catalysts are unstable, difficult to be synthesized, and difficult in exhibiting their original characters after supported on carrier. All of these difficulties largely limit the applications and development of single-site catalysts. In addition to the aforementioned challenge, a large number of expensive cocatalysts such as alkyl aluminoxane (such as MMAO) is always needed to get high activity.

Compared to the single-site metallocene and non-metallocene catalysts, Ziegler-Natta catalyst are still the most important catalyst now. The main reason is closely related to their stability, simple preparation and low cost. However, because of the character of having multi active sites in Ziegler-Natta catalyst, the polymer structure can not be controlled well when Ziegler-Natta catalyst is used. In recent years, by using advanced Ziegler-Natta catalysts and chemical technology processes, polyolefin materials with excellent performance can be produced. For example: U.S. Pat. No. 5,459,116 discloses a kind of olefins polymerization catalyst. The catalyst is prepared by directly reacting a magnesium compound of liquid phase having no reducing power with a titanium compound of liquid phase in the presence of at least one electron donor, which contains at least one hydroxyl group. Superior in activity as well as production yield in polymerizing olefins, the catalyst is capable of not only providing the polymer with high stereoregularity but also improving the bulk density of the polymer, especially polyethylene; U.S. Pat. Nos. 5,106,807 and 4,330,649 disclose the activity of catalysts and polymer molecular weight can be controlled by the addition of ester compounds; CN 1189487C (PCT/KR2000/001549) provides a method to prepare ethylene homopolymers and copolymers with narrow molecular weight distributions 3.6-4.3; Terano reported Ziegler-Natta catalysts supported either on surface functionalized SiO₂ or on ethylene/propylene/diene elastomers (EPDM). The molecular weight distribution of polyethylenes varied from narrow to broad (1.6-30) by solely changing the type of Al-alkyl cocatalyst. This is the narrowest molecular weight distribution obtained by Ziegler-Natta catalyst (Terano, M. Catalysis Commun. 2003, 4, 657-662; Macromol. Chem. Phys. 1998, 199, 1765), however, either the activity of catalyst or the polymer molecular weight decreased significantly.

### SUMMARY OF THE INVENTION

The purpose of the invention is to provide a new class of organic compounds containing heteroatoms.

The purpose of the invention is also to provide the application of the organic compounds as electronic donors in the preparation of the single-site Ziegler-Natta catalyst.

The purpose of the invention is also to provide a new class of single-site Ziegler-Natta catalysts and their preparation methods.

The purpose of the invention is to provide the usage of the catalysts and the catalysts systems. The catalysts and the catalysts systems are highly active to catalyze the ethylene polymerization or copolymerization with α-olefin of C₃-C₁₈, with good control of the polymer molecular weight and well comonomer distribution. The molecular weight distribution (PDI) of the obtained polymer is narrow (PDI 1.6 to 5.0).

The present invention provides a new class of organic compounds containing heteroatoms and their applications as electron donors in the preparation of single-site Ziegler-Natta catalyst, along with magnesium compound and metal compound or/and supporter. The organic compounds may be easily synthesized in high yields under mild conditions by refluxing the corresponding 1,3-diketone derivatives with amine derivatives in organic solvents for 2-48 hours.

Upon activation with cocatalysts such as alkyl aluminum, the prepared single-site Ziegler-Natta catalysts are highly active for ethylene polymerization or copolymerization with α-olefin of C₃-C₁₈, with the highest activity of ethylene polymerization up to 18000 g polymer/g catalyst; the incorporation ratio of comonomer such as 1-hexene can be higher than 2.0 mol %. The molecular weight distribution of the resulting polymer is narrow (PDI 1.6 to 5.0), and the structure of the polymer is controllable. All of the distinguish characters make the catalyst suitable for commercialization.

The structure of the organic compounds containing heteroatoms is shown below (I), and in organic solvents which may be a mixture of two tautomerisms I and II:

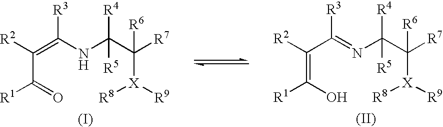

### DETAILED DESCRIPTION OF THE INVENTION

The organic compounds containing heteroatoms provided in the present invention are showed below:

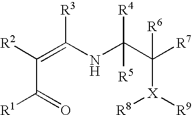

in the compound, R¹ and R², respectively, is H, hydrocarbyl of C₁-C₃₀, substituted hydrocarbyl of C₁-C₃₀, aryl group of C₅-C₅₀, or substituted aryl group of C₅-C₅₀, while these groups may be same or different;

R³, R⁴, R⁵, R⁶, R⁷, R⁸, and R⁹, respectively, is H, hydrocarbyl group of C₁-C₃₀, substituted hydrocarbyl group of C₁-C₃₀, aryl group of C₅-C₅₀, or substituted aryl group of C₅-C₅₀, while these groups may be same or different, of which, R⁴, R⁵ with R⁶ or R⁷, R⁶ with R⁸ or R⁹, R⁷ with R⁸ or R⁹ may form a bond or form a cycle;

X is O, N, S, Se or P;

when X is O, S or Se, there is only one group R⁸ or R⁹ on X;

the aryl group is phenyl, naphthyl or other heteroaromatic group;

the substituted hydrocarbyl group or substituted aryl group is the group substituted with hydrocarbyl, halogen, carbonyl group, ester group, group containing silicon, group containing oxygen atom —OR¹⁰, group containing sulfur atom —SR¹¹ or —S(O)R¹², group containing nitrogen atom —NR¹³R¹⁴ or —N(O)R¹⁵R¹⁶, or group containing phosphorous atom —PR¹⁷R¹⁸ or —P(O)R¹⁹R²⁰, group containing selenium atom —SeR¹¹ or —Se(O)R¹²;

R¹⁰, R¹¹, R¹², R¹³, R¹⁴, R¹⁵, R¹⁶, R¹⁷, R¹⁸, R¹⁹, R²⁰, R²¹ or R²² is substituted hydrocarbyl group of C₁-C₃₀, aryl group of C₅-C₅₀, of them, R¹³ and R¹⁴, R¹⁵ and R¹⁶, R¹⁷ and R¹⁸, R¹⁹ and R²⁰ can link to one another to form covalent bond or to form a ring;

The organic compound containing heteroatom in the present invention has a structure of following general formula (I), and which can be a mixture of I and II in organic solvents:

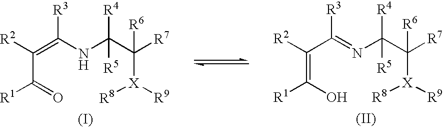

R¹-R⁹ are the groups as aforementioned.

Examples representative of compound I include ED01-ED44, and it needs to emphasize that the compound provided in present invention is not limited to these examples:

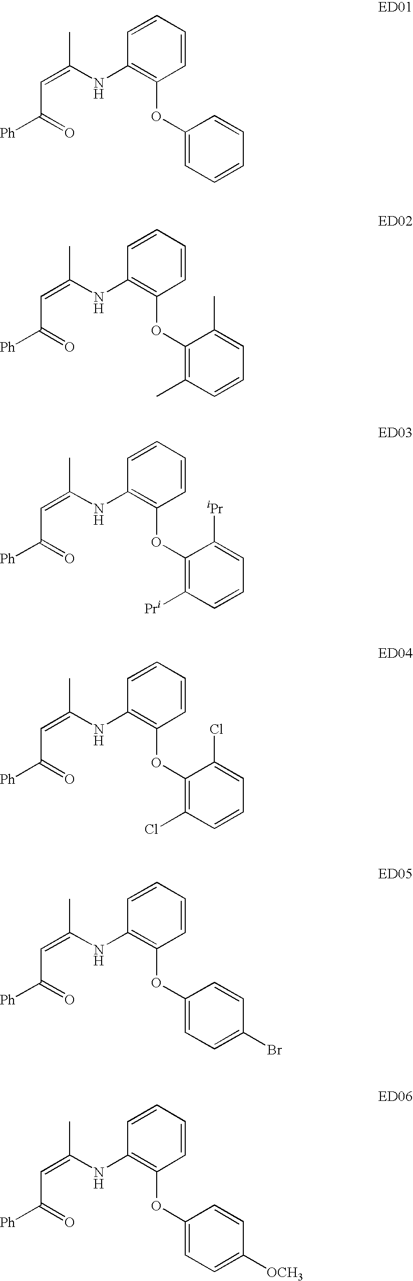

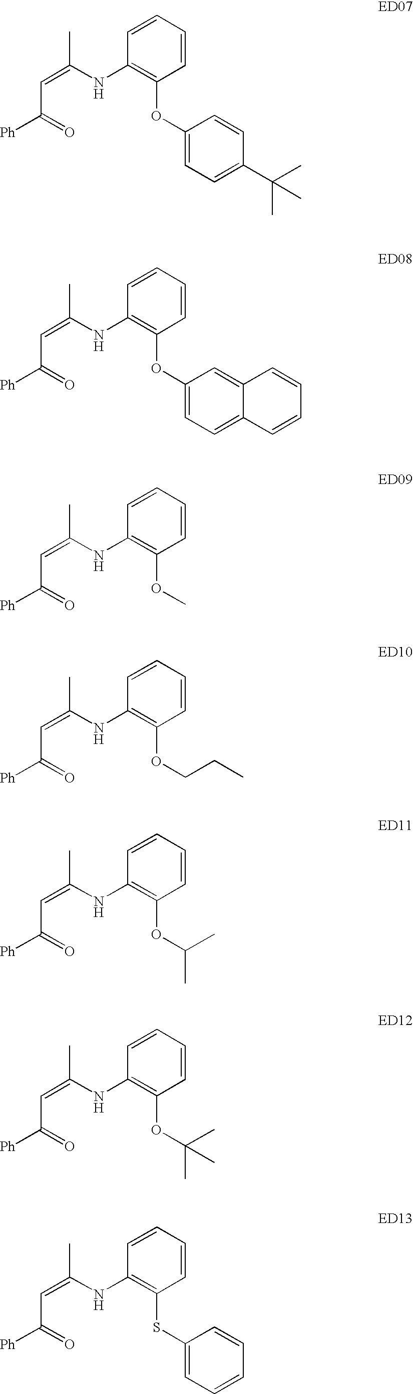

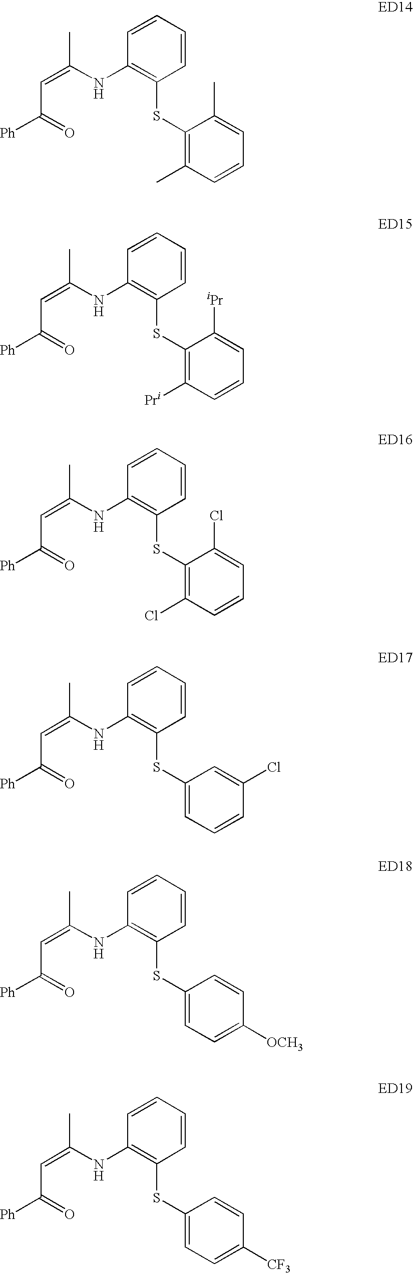

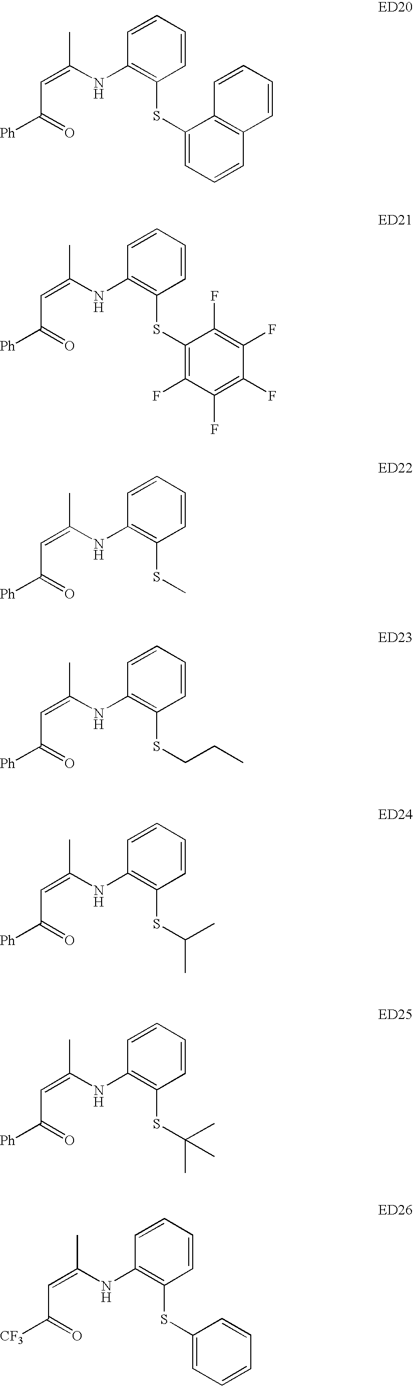

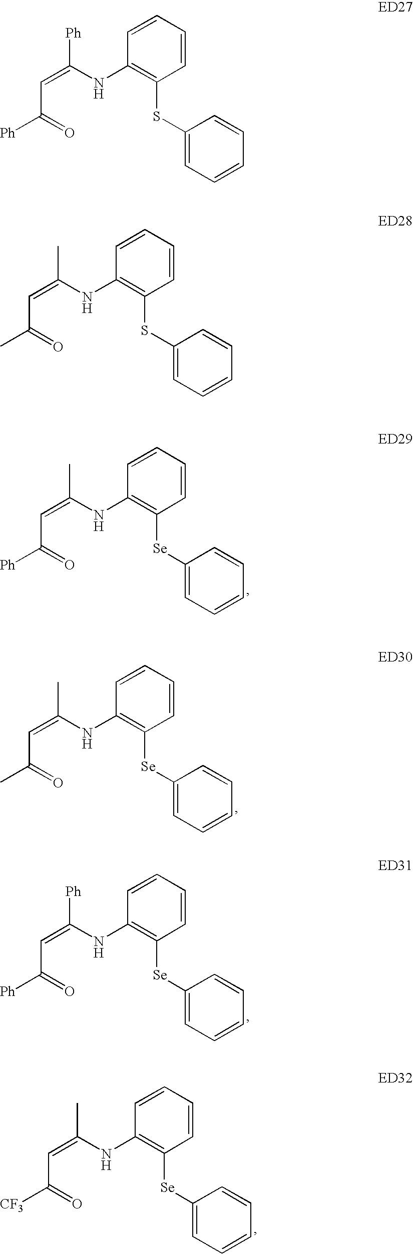

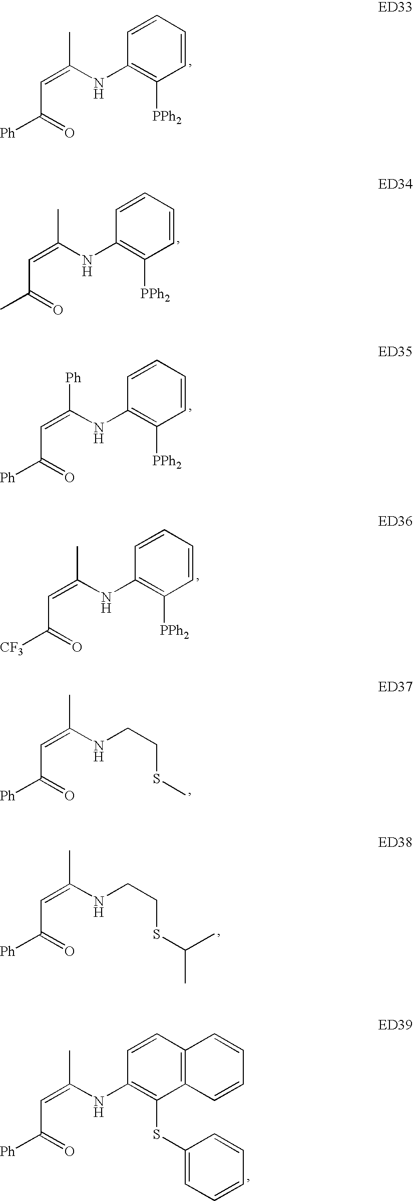

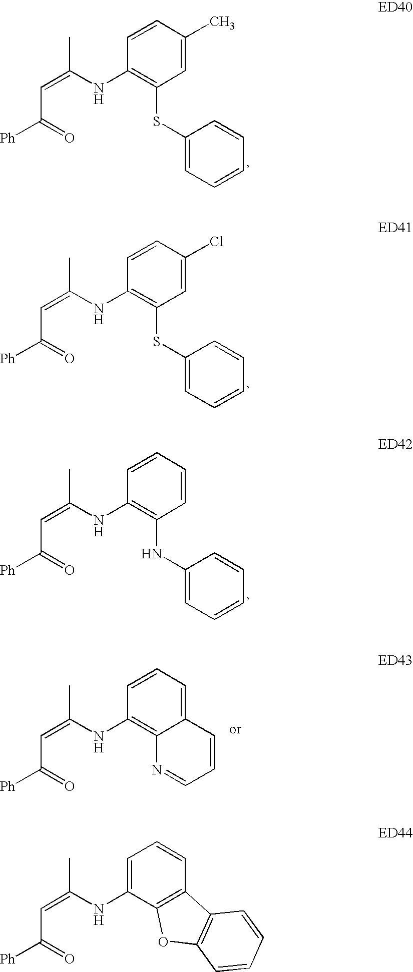

One organic compound or a mixture of two or more of compounds mentioned above can be used as electron donor (ED) in preparing single-site Ziegler-Natta catalyst.

### Preparation of the Organic Compound Containing Heteroatom

The organic compound can be synthesized according to literature methods (Hu W.-Q. et. al., Organometallics 2004, 23, 1684-1688; Wang, C. et. al. Macromol. Rapid Commun. 2005, 26, 1609-1614).

In the present invention, the compound is prepared in organic solvent by mixing the diketone derivative (III) with amine derivative (IV) in the presence of catalyst as showed below. The mixture is refluxed for 2-48 hrs, and after removing the solvent, the residue is purified by recrystallization in alcohol solvent to get compound (I).

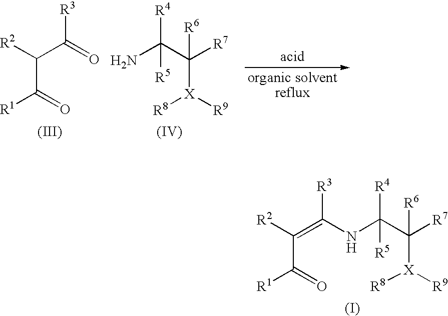

The catalyst in the reaction is formic acid, acetic acid, TsOH, or the other organic acid; the organic solvent is methanol, ethanol, or others, and anhydrous ethanol is optimal;

the molar ratio of diketone, amine, and catalyst is 1-1.5:1:0.01-0.1;

diketone is described by formula (III):

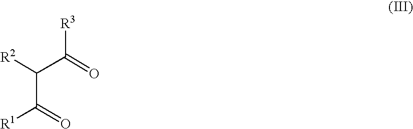

amine can be described by formula (IV):

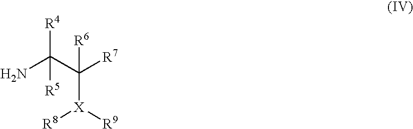

R¹-R⁹ are the groups as those mentioned above.

In the present invention, the single-site Ziegler-Natta catalyst is made of magnesium compound, supporter, metal complex, and the organic compound containing heteroatom, and the content of metal is in the range of 0.1-15 wt %.

The magnesium compound can be magnesium halide, alkyl magnesium, alkoxy magnesium halide, alkoxy magnesium or magnesium halide coordinate alcohol. Of the above named magnesium compound, a mixture of two or more may also be used; the magnesium halide or alkyl magnesium is the optimum.

The supporter of the single-site Ziegler-Natta catalyst can be organic material, metal oxides of group 2, 4, 13, and 14, clay, or molecular sieve. The metal oxides may be Al₂O₃, SiO₂, or a mixture of two or more metal oxides.

The “metal complex” can be represented by the formula (V):

MLₐ (V)

### Wherein:

a is 3, 4, 5 or 6;

L is selected from halogen atom, hydrocarbyl group of C₁-C₃₀, group containing oxygen atom, group containing nitrogen atom; each L in the formula may be same or different, and they may link to one another to form bonds or form a ring;

the halogen atom is F, Cl, Br, or I;

the group containing oxygen atom selected from alkoxy —OR²³, tetrahydrofuran or diethyl ether; The group containing nitrogen atom selected from —NR²⁴R²⁵ or —N(O)R²⁶R²⁷;

R²³-R²⁷, respectively, is H, hydrocarbyl group of C₁-C₃₀, or aryl group of C₅-C₅₀; these groups may be same or different, and R²⁴ with R²⁵, R²⁶ with R²⁷ may form a bond or to form a ring;

M is a transition metal of group 4 to group 6, preferable to titanium, zirconium, chromium, and vanadium.

Examples of the “metal complex” include titanium compound, zirconium compound, chromium compound, or vanadium compound, where Titanium compound may be tetrahalogenated titanium or tetrahalogenated titanium coordinated with THF or Et₂O, preferable to TiCl₄, TiCl₄(THF)₂; or alkoxy trihalogenated titanium, the preferable are Ti(OCH₃)Cl₃, Ti(OC₂H₅)Cl₃ or Ti(OC₂Hs)Br₃; or alkoxy dihalogenated titanium, the preferable are Ti(OCH₃)₂Cl₂, Ti(OC₂H₅)₂Cl₂; or alkoxy halogenated titanium, preferable to Ti(OCH₃)₃Cl, Ti(OC₂Hs)₃Cl; or tetraalkoxy titanium, tetraamido titanium or tetraalkyl titanium; Zirconium compound prefer ZrCl₄ or tetraamido zirconium; Chromium compound prefer CrCl₃ or CrCl₃(THF)₃; Vanadium compound is VCl₅, VCl₃(THF)₃ or VCl₃(PMe)₃. The more preferable “metal complex” is TiCl₄, TiCl₄(THF)₂, Ti(NMe₂)₄, Ti(NEt₂)₄, Ti(CH₂Ph)₄, ZrCl₄, Zr(NMe₂)₄, Zr(NEt₂)₄, CrCl₃, CrCl₃(THF)₃, VCl₃, or VCl₃(THF)₃. The most preferable “metal complex” is TiCl₄, TiCl₄(THF)₂, Ti(CH₂Ph)₄, ZrCl₄, CrCl₃, CrCl₃(THF)₃ or VCl₃(THF)₃.

### Preparation of the Single Site Ziegler-Natta Catalyst

In the present invention, the organic compound containing heteroatom is used effectively as an electron donor (ED) to prepare a single site Ziegler-Natta catalyst by the following procedure:

(1) pretreating an organic or an inorganic solid or a mixture of them by heating;

(2) dissolving magnesium compound in THF to form a solution at room temperature to 70° C.;

(3) to the aforesaid solution (2) was added the pretreated solid (supporter), metal complex and the electron donor, the resulting mixture was kept for several hours under certain temperature, and then removing the solvent, and the residue was washed with inert hydrocarbon solvent and was dried under reduced pressure to provide single-site Ziegler-Natta catalyst.

In step (1), the solid, which is used as a supporter, is treated at 30-1000° C. for 1-24 hrs under inert atmosphere and reduced pressure; and the optimal supporter is silica with particle size of 1-50 μm, specific surface area of 100-300 m²/g, pore volume of 0.5-3 mL/g, and an average pore diameter of 10-50 nm.

In step (2), the ratio between magnesium compound and THF is 1 g:1-100 mL, preferably 1 g:20-80 mL.

In step (3), the weight ratio between magnesium compound and supporter is 1:0.1-20, preferably 1:0.5-10; the mole ratio of magnesium compound and metal complex is 0.5-100:1, preferably 0.5-50:1; the mole ratio of electron donor (ED) and metal complex is 0.01-10:1, preferably 0.1-5:1; the reaction temperature is room temperature to 100° C., preferably 50-70° C.; reaction time is 2-48 hrs, preferably 4-24 hrs.

In step (3), the inert hydrocarbon solvent is hydrocarbon of C₅-C₁₀ or arene of C₆-C₈, which is selected from pentane, hexane, decane, heptane, octane or toluene, preferably hexane or toluene.

In step (3), it is workable to treat magnesium compound with metal complex for 2-48 hrs at room temperature to 100° C. first, then with the pretreated supporter, and finally with electron donor for 2-48 hrs at room temperature to 100° C. After removing the solvent, the residue was washed with inert hydrocarbon solvent and dried to provide Ziegler-Natta catalyst; the procedure can also be carried out by the following sequence: treating magnesium compound with a supporter for 2-48 hrs at room temperature to 100° C. to get a composite supporter which then react with a solution of an electronic donor and metal complex for 2-48 hrs at room temperature to 100° C., and by the same treatment mentioned above to provide the desired catalyst.

In the present invention, the solvents used during preparing single site Ziegler-Natta catalyst are treated to remove water and oxygen strictly and all manipulations were performed under inert atmosphere using standard Schlenk techniques which would not be described again in the following examples.

The catalyst in the present invention is suitable for ethylene polymerization, ethylene/α-olefin copolymerization, and ethylene/cycloolefin copolymerization. Alkyl aluminum, alkyl aluminoxane, or a mixture of two or more of them is used as cocatalyst in the polymerization process. A suitable cocatalyst selected from AlEt₃, Al(i-Bu)₃, AlEt₂Cl, Al(n-Hex)₃, MAO, EAO, MMAO, or a mixture of two or more of them, preferably AlEt₃, MMAO; the suitable mole ratio of Al/Ti is 20-1000, preferably 20-500; the useful α-olefins in the invention are C₃-C₂₀ such as propene, 1-butene, 1-hexene, 1-octene, 1-heptene, 4-methyl-1-petene, 1-decene, 1-undecene, 1-dodecene, 1-tetradecene, 1-hexadecene, 1-octadecene; the cycloolefins are cyclopetene, cyclohexene, norbornene or their derivatives. Either α-olefins and cycloolefins used in the present polymerization can be substituted by hydroxyl group, carboxyl group, ester group, or amine group.

The polymerization can be run in slurry process or gas process.

In the case of slurry polymerization process, the polymerization is generally performed at 80-120° C. under a total pressure of 0.1-10 MPa with 0-1.0 MPa hydrogen pressure; the polymerization may be carried out under supercritical or subcritical state with inert solvent such as propane, isobutane or hexane as solvent; both autoclave and loop reactor are useful.

In the case of gas polymerization process, the polymerization is generally conducted under a total pressure of 0.1-10 MPa at 40-100° C. in gas fluidized bed or gas autoclave.

The metal mass content of the produced single-site Ziegler-Natta catalyst is measured by ICP-AES, OPTRMA-3000 inductively coupled plasma atomic emission spectrometry.

Molecular weight and molecular weight distribution of the polymers are determined by Waters Alliance GPC2000 (differential refractive index detector) at 135° C. and 1,2,4-trichlorobenzene as eluent, polystyrene as a reference sample.

The ¹³C NMR of the polymer was determined by Varian XL-300 MHz nuclear magnetic resonance spectrometer at 110° C. in d₄-o-dichlorobenzene. And the incorporation of the comonomer is calculated by the literature method (J. C. Randall, JMS-Rev. Maromol. Chem. Phys. 1989, C29(2&3), 201-317).

### BRIEF DESCRIPTION OF THE FIGURES

FIG. 1 is the X-ray of compound ED14 in example 9.

FIG. 2 is the ¹³C NMR of the ethylene/1-hexene copolymer in example 78.

### EXAMPLES

### Example 1

### Synthesis of Electron Donor (ED)

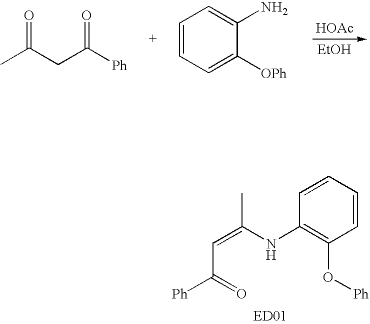

To a solution of 1-phenyl-1,3-butanedione (42.0 mmol) and 2-phenoxybenzenamine (40.0 mmol) in anhydrous ethanol (30 mL) was added acetic acid (3 mL). After refluxing for 30 hrs, the resulting mixture was cooled to 0° C. and filtered, the solid was washed with cool ethanol and dried to give ED01 as yellow solid. Yield 5.534 g (42%). ED01: ¹H NMR (300 MHz, CDCl₃): δ (ppm) 12.82 (s, 1H), 7.87-7.84 (m, 2H), 7.44-6.91 (m, 12H), 5.86 (s, 1H), 2.12 (s, 3H); ¹³C NMR (75 MHz, CDCl₃): δ (ppm) 188.83, 162.14, 155.35, 150.68, 139.91, 130.85, 130.29, 129.68, 128.52, 128.18, 127.06, 127.01, 126.87, 124.03, 119.77, 119.53, 94.65, 20.34.

### Example 2-7

Examples 2-7 provide some examples of the prepared electron donor (ED)

The same procedure as that for the preparation of ED01 was used. These compounds were prepared with the corresponding diketone derivatives and amine derivatives. The characterization data of the ED are showed as following:

### Example 2

ED02: ¹H NMR (300 MHz, CDCl₃): δ (ppm) 13.02 (s, 1H), 7.94-7.91 (m, 2H), 7.44-6.98 (m, 9H), 6.45-6.42 (m, 1H), 5.96 (s, 1H), 2.34 (s, 3H), 2.15 (s, 6H); ¹³C NMR (75 MHz, CDCl₃): δ (ppm) 188.76, 162.72, 151.32, 151.06, 140.11, 131.22, 130.73, 128.99, 128.16, 127.36, 127.09, 126.91, 126.44, 125.25, 121.29, 113.45, 94.43, 20.48, 16.32.

### Example 3

ED05: ¹H NMR (300 MHz, CDCl₃): δ (ppm) 12.82 (s, 1H), 7.88-7.85 (m, 2H), 7.44-6.86 (m, 11H), 5.87 (s, 1H), 2.13 (s, 3H); ¹³C NMR (75 MHz, CDCl₃): δ (ppm) 188.86, 162.13, 155.96, 150.53, 139.91, 132.63, 130.85, 130.38, 128.19, 127.07, 127.02, 126.89, 124.13, 120.13, 119.69, 115.97, 94.68, 20.32.

### Example 4

ED06: ¹H NMR (300 MHz, CDCl₃): δ 12.82 (s, 1H), 7.88-7.84 (m, 2H), 7.42-7.38 (m, 5H), 7.10-6.84 (m, 6H), 5.86 (s, 1H), 3.72 (s, 3H), 2.09 (s, 3H).

### Example 5

ED07: ¹H NMR (300 MHz, CDCl₃): δ (ppm) 12.84 (s, 1H), 7.89-7.86 (m, 2H), 7.43-6.93 (m, 11H), 5.87 (s, 1H), 2.15 (s, 3H), 1.28 (s, 9H).

### Example 6

ED08: ¹H NMR (300 MHz, CDCl₃): δ (ppm) 12.91 (s, 1H), 7.85-7.79 (m, 5H), 7.42-7.26 (m, 11H), 5.85 (s, 1H), 2.17 (s, 3H); ¹³C NMR (75 MHz, CDCl₃): δ (ppm) 188.72, 162.35, 154.31, 151.14, 139.97, 134.14, 130.73, 130.29, 130.15, 129.91, 128.12, 127.69, 127.09, 127.04, 126.96, 126.82, 126.47, 124.77, 123.63, 119.78, 119.41, 114.20, 94.58, 20.37.

### Example 7

ED09: ¹H NMR (300 MHz, CDCl₃): δ 12.82 (s, 1H), 7.95-7.91 (m, 2H), 7.41-7.14 (m, 7H), 5.92 (s, 1H), 3.64 (s, 3H), 2.06 (s, 3H).

### Example 8

To a solution of 1-phenyl-1,3-butanedione (10.0 mmol) in CH₃OH (15 mL) was added 2-(phenylthio)benzenamine (10.0 mmol), and then was added formic acid (0.5 mL). After refluxing for 48 hrs, solvent was removed and the residue was cooled. The generated solid was collected and recrystallized from ethanol and dried to give ED13. Yield 1.8156 g (53%). ¹H NMR (300 MHz, CDCl₃): δ (ppm) 12.93 (s, 1H), 7.94-7.91 (m, 2H), 7.47-7.38 (m, 5H), 7.31-7.15 (m, 7H), 5.85 (s, 1H), 1.97 (s, 3H); ¹³C NMR (75 MHz, CDCl₃): δ (ppm) δ 188.84, 162.06, 139.94, 137.78, 133.95, 133.52, 132.55, 131.20, 130.83, 129.25, 127.78, 127.22, 127.14, 126.99, 126.88, 94.32, 20.06, IR: 3060, 1597, 1574, 1546, 1508, 1462, 1425, 1317, 1287, 1271, 1060, 760, 747, 732 cm⁻¹; LRMS-EI(m/z): 345 (M⁺), 91 (100); elemental analysis for C₂₂H₁₉NOS: C, 76.64; H, 5.63; N, 3.77.

### Example 9

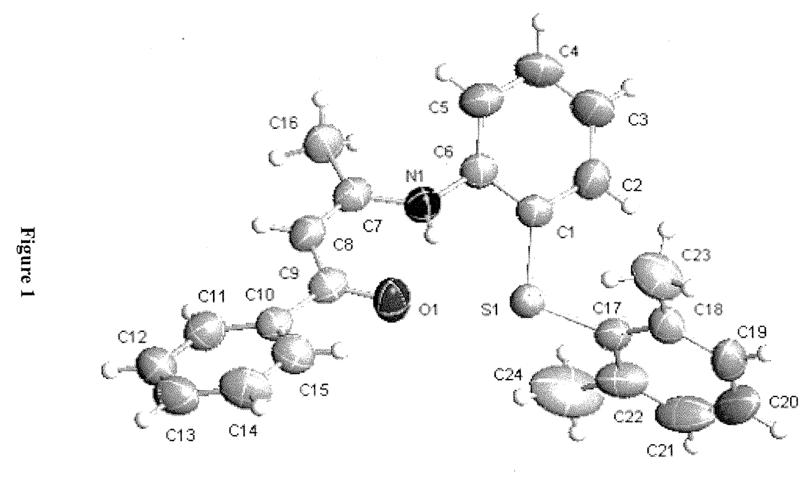

To a solution of 1-phenyl-1,3-butanedione (1.92 mmol) in anhydrous C₂H₅OH (7 mL) was added 2-(2,6-dimethylphenylthio)benzenamine (1.74 mmol), and then was added acetic acid (0.6 mL). After refluxing for 24 hrs, solvent was removed and the residue was cooled. The generated solid was collected and recrystallized from ethanol and dried to give ED14. Yield 0.4657 g (72%) ¹H NMR (300 MHz, CDCl₃): δ 12.84 (s, 1H), 7.99-7.96 (m, 2H), 7.46-7.43 (m, 3H), 7.25-7.01 (m, 6H), 6.46-6.43 (m, 1H), 5.99 (s, 1H), 2.40 (s, 6H), 2.06 (s, 3H); ¹³C NMR (75 MHz, CDCl₃): δ (ppm) 188.93, 163.21, 144.06, 139.91, 136.21, 135.14, 130.83, 129.45, 129.17, 128.54, 128.15, 127.50, 127.15, 124.86, 124.76, 93.95, 21.68, 20.01; IR: 3450, 3060, 2920, 1599, 1577, 1550, 1461, 1317, 1284, 747 cm⁻¹; LRMS-EI(m/z): 373 (M⁺), 105 (100); elemental analysis for C₂₄H₂₃NOS: C, 77.44; H, 6.18; N, 3.34. Molecular structure of ED14 is showed in FIG. 1.

### Example 10

To a solution of 1-phenyl-1,3-butanedione (1.16 mmol) in anhydrous C₂H₅OH (7 mL) was added 2-(2,6-diisopropylphenylthio)benzenamine (1.05 mmol), and then was added formic acid (0.2 mL). After refluxing for 8 hrs, solvent was removed and the residue was cooled. The generated solid was collected and recrystallized from ethanol and dried to give ED15 (0.3203 g, 71%): ¹H NMR (300 MHz, CDCl₃): δ 12.82 (s, 1H), 8.00-7.97 (m, 2H), 7.48-7.01 (m, 9H), 6.40-6.37 (m, 2H), 6.02 (s, 1H), 2.08 (s, 3H), 1.15-1.12 (d, J=7.2 Hz, 12H); ¹³C NMR (75 MHz, CDCl₃): δ (ppm) 188.98, 154.22, 139.96, 138.55, 130.87, 130.45, 128.22, 127.57, 127.50, 127.20, 126.78, 125.34, 124.55, 124.24, 93.89, 31.66, 24.17, 19.96; IR: 3060, 2960, 1597, 1557, 1461, 1319, 1284, 745 cm⁻¹; LRMS-EI (m/z): 430 (M⁺), 252 (100); elemental analysis for C₂₈H₃₁NOS: C, 78.29; H, 7.51; N, 3.07.

### Example 11

To a solution of 1-phenyl-1,3-butanedione (1.22 mmol) in anhydrous C₂H₅OH (10 mL) was added 2-(2,6-dichlorophenylthio)benzenamine (1.11 mmol), and then was added formic acid (0.5 mL). After refluxing for 20 hrs, solvent was removed and the residue was cooled. The generated solid was collected and recrystallized from ethanol and dried to give ED16 (0.3363 g, 73%): ¹H NMR (300 MHz, CDCl₃): δ 12.81 (s, 1H), 7.97-7.94 (m, 2H), 7.47-7.10 (m, 8H), 6.73-6.70 (m, 1H), 5.96 (s, 1H), 2.06 (s, 3H); ¹³C NMR (75 MHz, CDCl₃): δ (ppm) 189.00, 163.02, 141.70, 139.87, 136.02, 134.02, 130.86, 130.83, 130.29, 128.95, 128.17, 127.84, 127.57, 127.18, 126.83, 126.22, 94.16, 20.08; IR: 3420, 3060, 1600, 1578, 1553, 1426, 1317, 1283, 782, 750 cm⁻¹; LRMS-EI (m/z): 414 (M⁺), 105 (100); elemental analysis for C₂₂H₁₇Cl₂NOS: C, 63.54; H, 4.04; N, 3.20.

### Example 12-15

ED17-ED22 were prepared from the corresponding diketone derivatives and amine following the procedure of Example 11:

ED17: ¹H NMR (300 MHz, CDCl₃): δ 12.95 (s, 1H), 7.91-7.88 (m, 2H), 7.47-7.15 (m, 11H), 5.81 (s, 1H), 1.95 (s, 3H); ¹³C NMR (75 MHz, CDCl₃): δ (ppm) 189.00, 161.53, 139.83, 138.97, 136.57, 134.73, 132.99, 131.35, 130.89, 130.69, 130.08, 129.32, 128.44, 128.15, 127.39, 127.12, 126.99, 126.83, 94.59, 19.99.

### Example 13

ED18: ¹H NMR (300 MHz, CDCl₃): δ 12.89 (s, 1H), 7.97-7.94 (m, 2H), 7.44-7.40 (m, 5H), 7.13-6.86 (m, 6H), 5.91 (s, 1H), 3.80 (s, 3H), 1.99 (s, 3H); ¹³C NMR (75 MHz, CDCl₃): δ (ppm) 188.83, 162.59, 160.10, 139.92, 136.71, 136.21, 136.02, 130.85, 128.51, 128.18, 127.17, 127.03, 125.96, 122.36, 115.06, 109.71, 94.10, 55.32, 20.12.

### Example 14

ED21: ¹H NMR (300 MHz, CDCl₃): δ 12.86 (s, 1H), 7.95-7.92 (m, 2H), 7.48-7.20 (m, 7H), 5.95 (s, 1H), 2.00 (s, 3H); ¹³C NMR (75 MHz, CDCl₃): δ (ppm) 189.28, 162.20, 139.60, 138.08, 131.07, 130.53, 128.42, 128.25, 128.06, 127.61, 127.15, 94.40, 19.96.

### Example 15

ED22: ¹H NMR (300 MHz, CDCl₃): δ 12.82 (s, 1H), 7.94-7.90 (m, 2H), 7.41-7.13 (m, 7H), 5.91 (s, 1H), 2.47 (s, 3H), 2.04 (s, 3H

### Example 16

To a solution of benzoyl acetone (5.54 mmol) in anhydrous C₂H₅OH (5 mL) was added 1-(phenylthio)propan-2-amine (5.54 mmol), and then was added formic acid (0.5 mL). After refluxing for 36 hrs, solvent was removed and the residue was cooled. The generated solid was collected and recrystallized from ethanol and dried to give ED23 (1.7245 g, 68%). ¹H NMR (300 MHz, CDCl₃): δ 12.86 (s, 1H), 7.96-7.94 (m, 2H), 7.46-7.17 (m, 7H), 5.94 (s, 1H), 2.89-2.84 (t, J=7.2 Hz, 2H), 2.04 (s, 3H), 1.71-1.64 (m, 2H), 1.05-1.00 (t, J=7.5 Hz, 3H). ¹³C NMR (75 MHz, CDCl₃): 188.83, 162.45, 139.97, 137.53, 134.23, 130.83, 128.83, 128.17, 127.19, 126.85, 126.75, 125.89, 94.21, 34.64, 22.28, 20.23, 13.52; IR: 3060, 2962, 1598, 1574, 1548, 1515, 1461, 1432, 1317, 1280, 1195, 1064, 754, 708 cm⁻¹; LRMS-EI(m/z): 311 (M⁺), 105 (100); elemental analysis for C₁₉H₂₁NOS: C, 73.20; H, 6.81; N, 4.23.

### Example 17-20

ED24-ED27 were prepared from the corresponding diketone derivatives and amine following the procedure of example 16:

### Example 17

ED24: ¹H NMR (300 MHz, CDCl₃): δ 12.94 (s, 1H), 7.97-7.93 (m, 2H), 7.49-7.17 (m, 7H), 5.93 (s, 1H), 3.41-3.37 (m, 1H), 2.07 (s, 3H), 1.31-1.29 (d, J=6 Hz, 6H); ¹³C NMR (75 MHz, CDCl₃): δ (ppm) 188.67, 161.81, 139.95, 139.17, 132.57, 131.96, 130.75, 128.11, 127.10, 126.34, 126.20, 94.43, 37.50, 22.91, 20.34; IR: 3060, 2980, 1598, 1577, 1511, 1436, 1320, 1280, 758, 703, 673 cm⁻¹; LRMS-EI(m/z): 311 (M⁺), 105 (100); elemental analysis for C₁₉H₂₁NOS: C, 73.19; H, 6.74; N, 4.14.

### Example 18

ED25: ¹H NMR (300 MHz, CDCl₃): δ (ppm) 13.17 (s, 1H), 7.98-7.94 (m, 2H), 7.66-7.63 (m, 1H), 7.47-7.17 (m, 6H), 5.93 (s, 1H), 2.14 (s, 3H), 1.32 (s, 9H); ¹³C NMR (75 MHz, CDCl₃): δ (ppm) 188.53, 160.52, 143.05, 139.82, 130.80, 129.64, 128.16, 127.20, 125.20, 125.02, 95.17, 47.86, 30.84, 20.82; IR: 3060, 2980, 1596, 1577, 1555, 1456, 1321, 1280, 759 cm⁻¹; LRMS-EI(m/z): 325 (M⁺), 105 (100); elemental analysis for C₂₀H₂₃NOS: C, 73.73; H, 7.07; N, 3.95.

### Example 19

ED26: ¹H NMR (300 MHz, CDCl₃): δ (ppm) 12.35 (s, 1H), 7.34-7.17 (m, 8H), 5.44 (s, 1H), 1.91 (s, 3H); ¹³C NMR (75 MHz, CDCl₃): δ (ppm) 176.53, 167.93, 136.39, 134.25, 132.97, 132.34, 131.83, 129.41, 128.30, 128.09, 127.70, 127.25, 115.47, 90.81 (t), 19.92; IR: 3155, 2925, 2852, 1620, 1590, 1565, 1467, 1439, 1428, 1292, 1241, 1062, 861, 753, 745, 734 cm⁻¹; elemental analysis for C₁₇H₁₄F₃NOS: C, 60.68; H, 4.15; N, 3.95.

### Example 20

ED27: ¹H NMR (300 MHz, CDCl₃): δ (ppm) 12.91 (s, 1H), 7.99-6.41 (m, 18H), 6.08 (s, 1H); ¹³C NMR (75 MHz, CDCl₃): δ (ppm) 189.59, 160.50, 139.94, 139.68, 135.87, 134.50, 132.95, 131.91, 131.31, 129.61, 129.12, 128.91, 128.44, 128.27, 128.03, 127.53, 127.41, 127.36, 124.90, 124.46, 97.92; IR: 3051, 1545, 1480, 1438, 1330, 1282, 1207, 1050, 1022, 781, 754, 686 cm⁻¹; elemental analysis for C₂₇H₂₁NOS: C, 79.23; H, 5.18; N, 3.13.

### Example 21

To a solution of acetylacetone (10 mmol) in CH₃OH (15 mL) was added 2-(phenylthio)benzenamine (10 mmol), and then was added formic acid (1 mL). After refluxing for 24 hrs, solvent was removed and the residue was cooled. The generated solid was collected and recrystallized from ethanol and dried to give ED28 (1.8156 g, 52.6%). ¹H NMR (300 MHz, CDCl₃): δ (ppm) 12.34 (s, 1H), 7.35-7.26 (m, 5H), 7.19-7.11 (m, 4H), 5.15 (s, 1H), 2.09 (s, 3H), 1.84 (s, 3H); ¹³C NMR (75 MHz, CDCl₃): 6 (ppm) 196.30, 159.95, 137.83, 133.68, 132.36, 131.14, 129.20, 128.92, 127.71, 127.17, 126.85, 126.66, 126.33, 97.77, 29.15, 19.49. IR: 3058, 1575, 1500, 1462, 1439, 1377, 1355, 1275, 1186, 1063, 1024, 993, 921, 751, 691, 660 cm⁻¹; LRMS-ET(m/z): 283 (M⁺), 174 (100); elemental analysis for C₁₇H₁₇NOS: C, 72.09; H, 6.02; N, 4.78.

### Example 22-31

ED33-44 were prepared following the procedure showed in example 21.

### Example 22

ED33: ¹H NMR (300 MHz, CDCl₃): δ (ppm) 12.90 (s, 1H), 7.93-7.14 (m, 19H), 5.81 (s, 1H), 1.94 (s, 3H).

### Example 23

ED35: ¹H NMR (300 MHz, CDCl₃): δ (ppm) 12.87 (s, 1H), 7.95-7.11 (m, 24H), 6.35 (s, 1H)o

### Example 24

ED37: ¹H NMR (300 MHz, CDCl₃): δ 13.08 (s, 1H), 7.81-7.49 (m, 5H), 5.77 (s, 1H), 3.02 (t, 2H), 2.70 (t, 2H), 2.09 (s, 3H), 1.96 (t, 3H)o

### Example 25

ED38: ¹H NMR (300 MHz, CDCl₃): δ 13.28 (s, 1H), 7.81-7.49 (m, 5H), 5.77 (s, 1H), 3.02 (t, 2H), 2.88 (m, 1H), 2.70 (t, 2H), 1.95 (t, 3H), 1.25 (d, 6H)o

### Example 26

ED39: ¹H NMR (300 MHz, CDCl₃): δ 12.38 (s, 1H), 7.81-6.56 (m, 16H), 5.99 (s, 1H), 1.71 (t, 3H)o

### Example 27

ED40: ¹H NMR (300 MHz, CDCl₃): δ 12.38 (s, 1H), 7.81-7.49 (m, 5H), 7.44-7.22 (m, 5H), 6.75-6.14 (m, 3H), 5.99 (s, 1H), 2.35 (s, 3H), 1.71 (s, 3H)o

### Example 28

ED41: ¹H NMR (300 MHz, CDCl₃): δ 12.89 (s, 1H), 7.97-7.64 (m, 5H), 7.44-7.22 (m, 5H), 6.66-6.24 (m, 3H), 5.95 (s, 1H), 1.91 (s, 3H)o

### Example 29

ED42: ¹H NMR (300 MHz, CDCl₃): δ 12.38 (s, 1H), 9.77 (s, 1H), 7.81-7.49 (m, 5H), 7.33-6.98 (m, 5H), 6.61-6.21 (m, 4H), 5.99 (s, 1H), 1.71 (s, 3H)o

### Example 30

ED43: ¹H NMR (300 MHz, CDCl₃): δ 12.38 (s, 1H), 8.80 (s, 1H), 7.94-6.84 (m, 10H), 5.97 (s, 1H), 1.73 (s, 3H)o

### Example 31

ED44: ¹H NMR (300 MHz, CDCl₃): δ 12.40 (s, 1H), 7.98-6.39 (m, 12H), 5.95 (s, 1H), 1.75 (s, 3H).

### Example 32

The synthesis of single site Ziegler-Natta catalyst In the present invention:

### (1) Thermo-Pretreatment of the Supporter

ES70 silica (product of Ineos company) is calcinated under nitrogen atmosphere at 200° C. for 2 hrs and then for 4 hrs at 400° C., after that it is cooled under nitrogen atmosphere to provide supporter ES70.

### (2) The Synthesis of Single-Site Ziegler-Natta Catalyst

### Method One:

A solution of anhydrous MgCl₂ (1.0 g) in tetrahydrofuran (THF for short, 40 mL) was stirred at 60° C. for 2 h; then to the solution was added TiCl₄ (3.4 mmol) and the reaction mixture was heated at 60° C. for 4 h. Then the pretreated ES70 supporter (1.0 g) was added and the resulting mixture was heated for further 4 hrs at 60° C. To the mixture was added desired electron donor and the reaction system was maintained at 60° C. for another 12 hrs. Then, the solvent was removed under reduced atmosphere, and the residue was washed with hexane (3×20 mL) and then dried under vacuum to provide a fluid brown powder. Ti content: 3.20 wt-%.

### Method Two:

To tetrahydrofuran (THF for short, 40 mL) was added anhydrous MgCl₂ (1.0 g) and the resulting suspension was stirred for 2 hrs at 60° C. to get MgCl₂ dissolved totally. To the resulting solution was added silica (1.7 g) and the mixture was stirred for 1 h. Hexane (40 mL) was added and then the reaction system was cooled to room temperature under stirring. After filtration, the obtained solid was dried under vacuum to provide a composite supporter.

To a solution of TiCl₄(THF)₂ in dichloromethane (2 mL) was added a solution of electronic donor ED01 in dichloromethane (2 mL), and the resulting solution was added to the composite supporter (0.77 g) with stirring. Removing the solvent under vacuum to provide Ziegler-Natta catalyst as fluid brown powders.

### Example 33-69

The following examples are the synthesis of the Ziegler-Natta catalyst according to the same procedure of example 32 (Table 1).

TABLE 1

| | | Anhydrous | THF | TiCl₄ | supporter | | Ti |
|---|---|---|---|---|---|---|---|
| Example | Catalyst | MgCl₂ (g) | (mL) | (mmol) | (g) | ED (mmol) | (wt-%) |
| 33 | SC02 | 1.0 | 40 | 3.4 | ES70(1.0) | ED02(3.5) | 4.61 |
| 34 | SC03 | 1.0 | 40 | 3.4 | ES757(1.0) | ED02(3.5) | 4.56 |
| 35 | SC04 | 1.0 | 40 | 3.4 | Grace955(1.0) | ED02(3.5) | — |
| 36 | SC05 | 1.2 | 50 | 3.4 | ES70(1.0) | ED05(3.5) | 4.35 |
| 37 | SC06 | 1.2 | 50 | 3.4 | ES70(1.0) | ED06(4.0) | — |
| 38 | SC07 | 0.9 | 40 | 3.4 | ES70X(1.0) | ED07(6.8) | 3.75 |
| 39 | SC08 | 1.2 | 50 | 3.4 | ES70Y(1.1) | ED08(4.0) | — |
| 40 | SC09 | 1.0 | 40 | 3.4 | ES70(1.0) | ED09(4.0) | 4.65 |
| 41 | SC11 | 1.0 | 40 | 3.4 | ES70(1.0) | ED13(4.1) | 4.50 |
| 42 | SC12 | 1.1 | 40 | 1.7 | ES70(1.3) | ED13(3.4) | 1.68 |
| 43 | SC13 | 0.5 | 20 | 3.4 | ES70(1.0) | ED13(4.1) | 1.09 |
| 44 | SC14 | 1.0 | 60 | 6.8 | ES70(2.0) | ED13(8.2) | 5.65 |
| 45 | SC15 | 1.0 | 40 | 3.0 | ES70(1.0) | ED13(1.5) | 4.59 |
| 46 | SC16 | 2.0 | 60 | 3.2 | ES757(2.0) | ED13(3.8) | — |
| 47 | SC17 | 0.5 | 30 | 1.7 | ES70(0.5) | ED14(2.6) | — |
| 48 | SC18 | 0.5 | 30 | 1.7 | ES70(0.5) | ED15(2.6) | 4.45 |
| 49 | SC19 | 0.5 | 30 | 1.7 | ES70(0.5) | ED16(2.6) | — |
| 50 | SC20 | 0.5 | 30 | 1.7 | ES70(0.5) | ED17(2.6) | 4.35 |
| 51 | SC21 | 0.5 | 30 | 1.7 | ES70(0.5) | ED18(2.6) | 4.37 |
| 52 | SC22 | 0.5 | 30 | 1.7 | ES70(0.5) | ED21(2.6) | 3.95 |
| 53 | SC23 | 0.5 | 30 | 1.7 | ES70(0.5) | ED22(2.6) | — |
| 54 | SC24 | 0.5 | 30 | 1.7 | ES70(0.5) | ED23(2.6) | 4.43 |
| 55 | SC25 | 0.5 | 30 | 1.7 | ES70(0.5) | ED24(2.6) | — |
| 56 | SC26 | 0.5 | 30 | 1.7 | ES70(0.5) | ED25(2.6) | 4.53 |
| 57 | SC27 | 1.0 | 60 | 6.8 | ES70(2.0) | ED26(8.2) | — |
| 58 | SC28 | 1.0 | 60 | 6.8 | ES757(2.0) | ED27(8.2) | — |
| 59 | SC29 | 1.0 | 60 | 6.8 | ES70(2.0) | ED28(8.2) | — |
| 60 | SC30 | 1.0 | 60 | 0.8 | ES70(2.0) | ED33(1.8) | 0.82 |
| 61 | SC31 | 0.5 | 60 | 0.8 | ES757(3.0) | ED35(1.6) | 0.40 |
| 62 | SC32 | 1.0 | 40 | 3.4 | ES70(1.0) | ED37(4.1) | — |
| 63 | SC33 | 1.0 | 40 | 3.4 | ES70(1.0) | ED38(4.1) | — |
| 64 | SC34 | 1.0 | 40 | 3.4 | ES70(1.0) | ED39(4.1) | — |
| 65 | SC35 | 1.0 | 40 | 3.4 | ES70(1.0) | ED40(4.1) | — |
| 66 | SC36 | 1.0 | 40 | 3.4 | ES70(1.0) | ED41(4.1) | — |
| 67 | SC37 | 1.0 | 40 | 3.4 | ES70(1.0) | ED42(4.1) | 4.10 |
| 68 | SC38 | 1.0 | 40 | 3.4 | ES70(1.0) | ED43(4.1) | 3.67 |
| 69 | SC39 | 1.0 | 40 | 3.4 | ES70(1.0) | ED44(4.1) | — |

### Example 70-74

The following examples are synthesis of the Ziegler-Natta catalyst containing electron donor according to the same procedure of example 32 (Table 2).

TABLE 2

| | Catalyst | | electronic | Metal content |
|---|---|---|---|---|
| example | number | Metal complex | donor (mmol) | (wt-%) |
| 70 | SC40 | TiCl₄(THF)₂ | ED13(3.5) | 4.42 |
| 71 | SC41 | ZrCl₄ | ED13(3.5) | 3.36 |
| 72 | SC42 | CrCl₃ | ED27(3.5) | — |
| 73 | SC43 | CrCl₃(THF)₃ | ED33(3.5) | 3.41 |
| 74 | SC44 | VCl₃(THF)₃ | ED35(4.0) | — |

### Example 75-103

The following examples are ethylene polymerization by slurry process: A 500 mL stainless-steel autoclave equipped with mechanical stirrer was dried under vacuum and then purged with nitrogen for three times and with ethylene for two times. Freshly distilled 180 g n-hexane (200 mL n-hexane+1.0 mL AlEt₃ (3.0 M in hexane)) was transferred to the reactor and the solution was stirred (rotate speed=150 rpm) at 60° C. Under nitrogen atmosphere, desired amount of comonomer (in the case of the copolymerization) and Ziegler-Natta catalyst (10 mg) were added in order then the pressure in autoclave was released. Raising the temperature of the solution to 80° C., and then ethylene gas was fed to get the pressure of autoclave to 1.0 MPa. After 5 min, the rotate speed was raised to 250 rpm and the temperature of water bath was raised to 85° C. for 1 h. The autoclave was cooled quickly to below 50° C., and the product was dried to get polymer as particle.

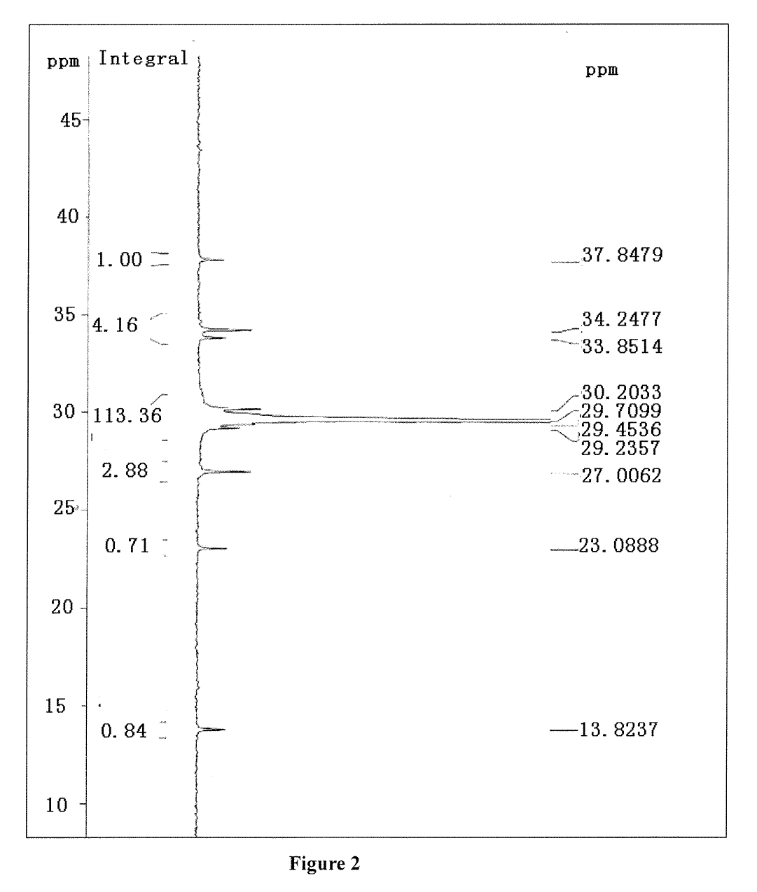

The detailed experimental conditions, catalytic activity (g polymer/g catalyst), polymer molecular weight M_{w} (g/mol), polymer molecular weight distribution (PDI) and the polymer bulk density (g/cm³), etc. were listed in Table 3. The ¹³C NMR of the ethylene/1-hexene copolymer obtained in example 78 was showed in FIG. 2.

TABLE 3

| | | | | | | | Comonomer |
|---|---|---|---|---|---|---|---|
| | | | Comonomer | Activity | M_{w} | | incorporation |
| Example | Catalyst | Comonomer | loading (g) | (g/g) | (10⁴ g/mol) | PDI | (mol-%) |
| 75 | SC01 | — | 0 | 1000 | 11.6 | 3.46 | — |
| 76 | SC02 | — | 0 | 930 | 12.7 | 3.35 | — |
| 77 | SC03 | 1-hexene | 10 | 1500 | 10.8 | 3.52 | 2.05 |
| 78 | SC04 | 1-hexene | 20 | 1200 | 10.3 | 3.56 | 1.04 |
| 79 | SC05 | — | 0 | 850 | — | — | — |
| 80 | SC06 | — | 0 | 1100 | — | — | — |
| 81 | SC07 | — | 0 | 1140 | — | — | — |
| 82 | SC08 | — | 0 | 740 | — | — | — |
| 83 | SC09 | 1-hexene | 10 | 1360 | 10.5 | | 1.95 |
| 84 | SC18 | — | 0 | 2000 | 16.7 | 2.45 | — |
| 85 | SC19 | — | 0 | 900 | 18.5 | 3.25 | — |
| 86 | SC20 | — | 0 | 3500 | 12.3 | 3.22 | — |
| 87 | SC21 | — | 0 | 1500 | 19.4 | 3.07 | — |
| 88 | SC22 | 1-hexene | 10 | 1840 | 15.2 | 3.20 | 2.03 |
| 89 | SC25 | 1-hexene | 10 | 4200 | 14.5 | 3.41 | 1.68 |
| 90 | SC26 | 1-hexene | 10 | 3700 | 13.8 | 3.35 | 1.81 |
| 91 | SC32 | — | 0 | 1660 | 14.0 | 3.56 | — |
| 92 | SC33 | — | 0 | 1200 | 11.2 | 1.81 | — |
| 93 | SC34 | — | 0 | 1500 | 1.3 | 3.20 | — |
| 94 | SC35 | — | 0 | 1430 | 1.5 | 3.11 | — |
| 95 | SC36 | — | 0 | 1700 | 10.3 | 3.69 | — |
| 96 | SC37 | — | 0 | 2100 | — | — | — |
| 97 | SC38 | — | 0 | 1500 | — | — | — |
| 98 | SC39 | — | 0 | 1250 | — | — | — |
| 99 | SC40 | — | 0 | 1650 | — | — | — |
| 100 | SC41 | — | 0 | 860 | — | — | — |
| 101 | SC42 | — | 0 | 1120 | — | — | — |
| 102 | SC43 | — | 0 | 1300 | — | — | — |
| 103 | SC44 | — | 0 | 1000 | — | — | — |

### Example 104-121

The following examples are ethylene (co)polymerization by slurry process:

A 2 L stainless-steel autoclave equipped with mechanical stirrer was dried under vacuum and then purged with nitrogen for three times and with ethylene for two times. Freshly distilled n-hexane (400 g) was transferred to the reactor and then the solution was stirred (rotate speed=150 rpm) at 60° C. Under nitrogen atmosphere, Ziegler-Natta catalyst (30 mg), n-hexane (200 g), and AlEt₃ (2.1 mL, 0.88 M in n-hexane solution) were added to a charging tank and were shaken sufficiently and then the charging tank were connected to the polymerization system. The solution in the charging tank was pressed into autoclave by nitrogen gas, and then the residual pressure in autoclave was released. At 70° C., ethylene gas was fed into the reactor to keep the pressure of the autoclave (the hydrogen has been pumped in first in the case of hydrogen modulation polymerization) to 0.8 MPa. After 5 min, the rotate speed was raised to 250 rpm and the temperature of water bath rose to 85° C. in the case of copolymerization, a certain amount of comonomers was added after the polymerization was ran for 20 min. After 2 h, the autoclave was cooled quickly to below 50° C. The product was vented and dried to get the polymer as particles.

The detailed experimental condition, catalytic activity (g polymer/g catalyst), polymer molecular weight M_{w} (g/mol), polymer molecular weight distribution (PDI), and the polymer bulk density (g/cm³), etc. were showed in Table 4.

TABLE 4

| | | | | | | | Comonomer |
|---|---|---|---|---|---|---|---|
| | | | Comonomer | Activity | M_{w} | | incorporation |
| Example | Catalyst | Comonomer | loading (g) | (g/g) | (10⁴ g/mol) | PDI | (mol %) |
| 104 | SC11 | — | 0 | 18000 | 30.2 | 2.18 | — |
| 105 | SC11 | 1-butene | 30 | 12500 | 31.5 | 2.65 | 0.15 |
| 106 | SC11 | 1-butene | 60 | 9500 | — | — | 0.34 |
| 107 | SC11 | 1-hexene | 30 | 11000 | 19.8 | 3.66 | 0.61 |
| 108 | SC11 | 1-hexene | 60 | 7400 | — | — | — |
| 109 | SC12 | — | 0 | 5300 | — | — | — |
| 110 | SC13 | — | 0 | 8600 | — | — | — |
| 111 | SC15 | — | 0 | 12600 | 25.4 | 3.12 | — |
| 112 | SC15 | 1-butene | 30 | 10500 | 19.0 | 3.45 | — |
| 113 | SC15 | 1-hexene | 30 | 7200 | — | — | — |
| 114 | SC15 | 1-hexene | 60 | 4700 | 17.6 | 3.19 | 0.26 |
| 115 | SC17 | — | 0 | 16000 | — | — | — |
| 116 | SC23 | — | 0 | 11600 | 38.4 | 2.05 | — |
| 117 | SC24 | 1-hexene | 30 | 12200 | 20.8 | 2.46 | 0.95 |
| 118 | SC28 | — | 0 | 3400 | — | — | — |
| 119 | SC29 | 1-hexene | 30 | 16300 | 21.5 | 2.32 | 0.87 |
| 120 | SC30 | — | 0 | 10400 | — | — | — |
| 121 | SC31 | — | 0 | 12000 | — | — | — |

1. Organic compounds containing heteroatoms have a formula of 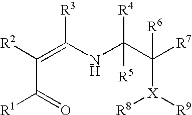
wherein
R¹ and R², respectively, is H, hydrocarbyl of C₁-C₃₀, substituted hydrocarbyl of C₁-C₃₀, aryl group of C₅-C₅₀, or substituted aryl group of C₅-C₅₀, these groups being same or different;
R³, R⁴, R⁵, R⁶, R⁷, R⁸, and R⁹, respectively, is H, hydrocarbyl group of C₁-C₃₀, substituted hydrocarbyl group of C₁-C₃₀, aryl group of C₅-C₅₀, or substituted aryl group of C₅-C₅₀, these groups being same or different, of which, R⁴, R⁵ with R⁶ or R⁷, R⁶ with R⁸ or R⁹, R⁷ with R⁸ or R⁹ may form a bond or a cycle;
X is O, N, S, Se, or P, and when X is O, S or Se, there is only one group, R⁸ or R⁹, on X;
the aryl group is phenyl, naphthyl, or other heteroaromatic group;
the substituted hydrocarbyl group or substituted aryl group is the group substituted with hydrocarbyl, halogen, group containing silicon, group containing oxygen atom —OR¹⁰, group containing sulfur atom —SR¹¹ or —S(O)R¹², group containing nitrogen atom-NR¹³R¹⁴ or —N(O)R¹⁵R¹⁶, or group containing phosphorous atom —PR¹⁷R¹⁸ or —P(O)R¹⁹R²⁰;
R¹⁰, R¹¹, R¹², R¹³, R¹⁴, R¹⁵, R¹⁶, R¹⁷, R¹⁸, R¹⁹, R²⁰, R²¹ or R²², respectively, is substituted hydrocarbyl group of C₁-C₃₀ or aryl group of C₅-C₅₀, R¹³ and R¹⁴, R¹⁵ and R¹⁶, R¹⁷ and R¹⁸, R¹⁹ and R²⁰ can link to one another to form covalent bond or to form a ring;
If R¹═Ph, then R²═H, R³═H or Me, X═S, and R⁸ or R⁹#≠Me or Ph;
If R¹═Ph or 4-C₁-C₆H₄—, then R²═H, R³═H or Me, and NHC(R⁴R⁵)C(R⁶R⁷)X(R⁸R⁹) is not 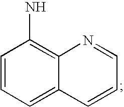
If R¹=Me or Ph, then R²═H, R³=Me or Ph, NHC(R⁴R⁵)C(R⁶R⁷)X(R⁸R⁹) is not 2-EtO—C₆H₄NH₂, 2-MeO—C₆H₄NH₂, or NH₂CH₂CH₂NH₂.

2. The organic compounds containing heteroatoms as recited in claim 1, which is a mixture of or either one of two tautomerism in organic solvents:

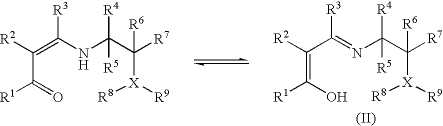

3. The organic compounds containing heteroatoms as recited in claim 1 having formulae of

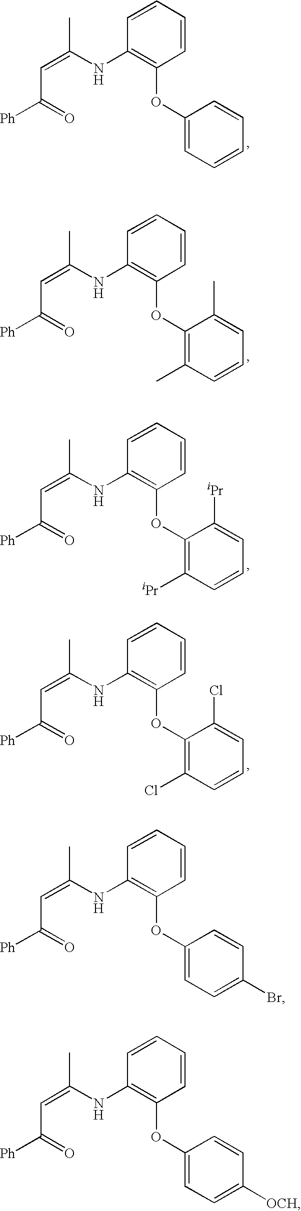

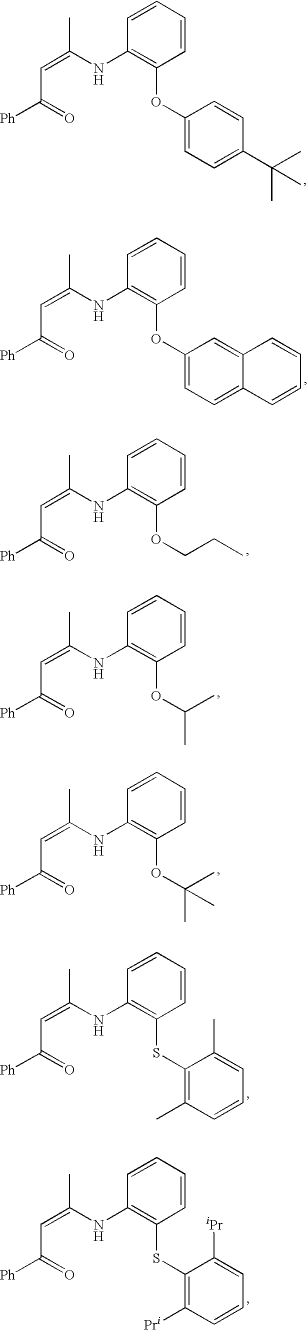

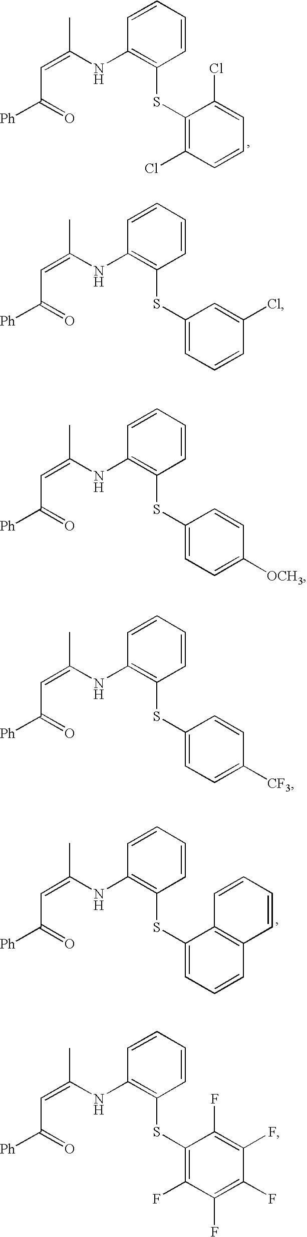

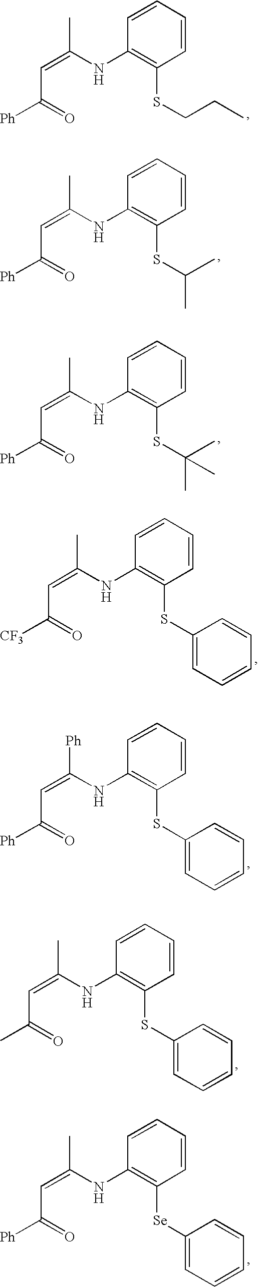

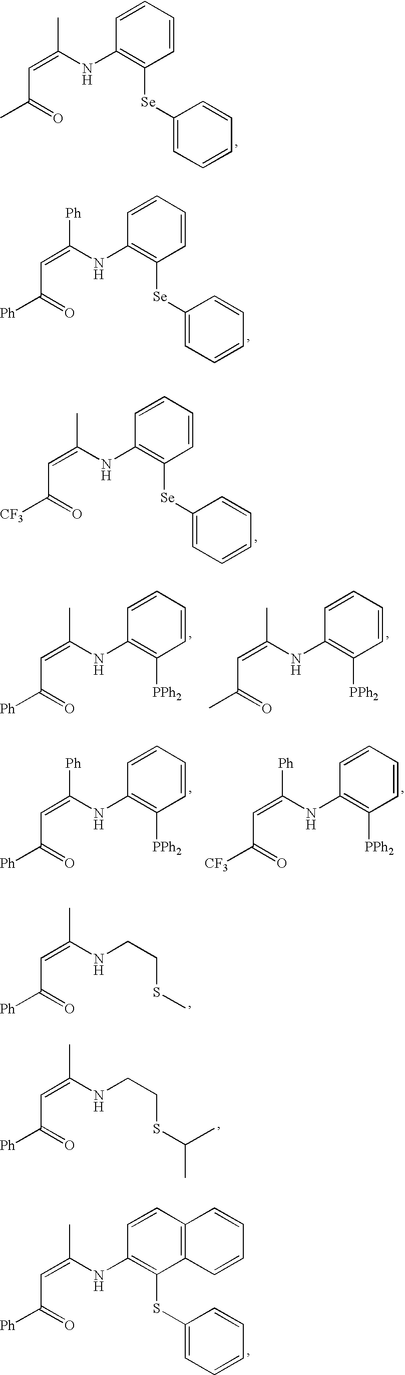

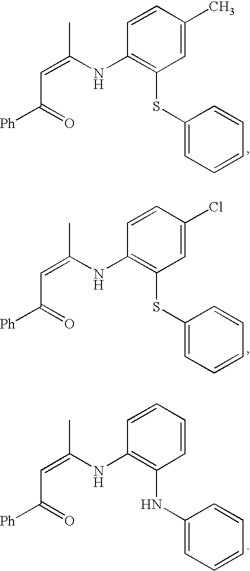

4. The organic compounds containing heteroatoms as recited in claim 1, wherein said compounds are prepared by refluxing a mixture of a diketone derivative and an amine derivative in an organic solvent in presence of a correspondence catalyst in a molar ratio of (1-1.5):1:(0.01-0.1) for 2-48 hrs,
wherein the diketone has a formula of 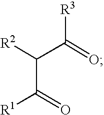
the amine has a formula of 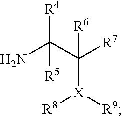
the catalyst is formic acid, acetic acid, TsOH, or another organic acid.

5. A method for preparing a single-site Ziegler-Natta catalyst, wherein organic compounds containing heteroatoms are used as electron donors, said organic compounds having a formula of 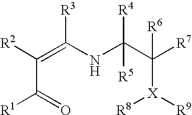
Wherein:
R¹ and R² respectively is H, hydrocarbyl of C₁-C₃₀, substituted hydrocarbyl of C₁-C₃₀, aryl group of C₅-C₅₀, or substituted aryl group of C₅-C₅₀, these groups being same or different;
R³, R⁴, R⁵, R⁶, R⁷, R⁸, and R⁹ respectively is H, hydrocarbyl group of C₁-C₃₀, substituted hydrocarbyl group of C₁-C₃₀, aryl group of C₅-C₅₀, or substituted aryl group of C₅-C₅₀, these groups being same or different, of which R⁴, R⁵ with R⁶ or R⁷, R⁶ with R⁸ or R⁹, R⁷ with R⁸ or R⁹ optionally form a bond or a cycle;
X is O, N, S, Se, or P, and when X is O, S or Se, there is only one group, R⁸ or R⁹, on X;
the aryl group is phenyl, naphthyl, or other heteroaromatic group;
the substituted hydrocarbyl group or substituted aryl group is the group substituted with hydrocarbyl, halogen, group containing silicon, group containing oxygen atom —OR¹⁰, group containing sulfur atom —SR¹¹ or —S(O)R¹², group containing nitrogen atom-NR¹³R¹⁴ or —N(O)R¹⁵R¹⁶, or group containing phosphorous atom —PR¹⁷R¹⁸ or —P(O)R¹⁹R²⁰;
R¹⁰, R¹¹, R¹², R¹³, R¹⁴, R¹⁵, R¹⁶, R¹⁷, R¹⁸, R¹⁹, R²⁰, R²¹ or R²² respectively is a substituted hydrocarbyl group of C₁-C₃₀ or aryl group of C₅-C₅₀, R¹³ and R¹⁴, R¹⁵ and R¹⁶, R¹⁷ and R¹⁸, R¹⁹ and R²⁰ may link to one another to form covalent bond or to form a ring;

6. The method for preparing a single-site Ziegler-Natta catalyst as recited in claim 5, wherein said catalyst is made of magnesium compound, supporter, metal complex, and the organic compounds containing heteroatom with a weight ratio of magnesium compound and supporter being 1:0.1-20, a molar ratio of magnesium compound and metal complex being 0.5-100:1, a molar ratio of the organic compound containing heteroatom and metal complex being 0.01-10:1, content of the metal being in a range of 0.1-15 wt %;
the magnesium compound of the single-site Ziegler-Natta catalyst is magnesium halide, alkyl magnesium, alkoxy magnesium halide, alkoxy magnesium, magnesium halide coordinate alcohol, or a mixture of two or more;
the supporter of the single-site Ziegler-Natta catalyst is an organic material or inorganic oxides containing the metal oxides of group 2, 4, 13 and 14, while the inorganic oxides is Al₂O₃, SiO₂, a mixture of oxides, clay, molecular sieve, or the oxide material provided by a gaseous metal oxide or silica compound through hydrolysis at high temperature;
the “metal complex” is represented by a formula of ML₃
Wherein:
a is 3 or 4;
L is a halogen atom, hydrocarbyl of C₁-C₃₀, group containing oxygen atom, or group containing nitrogen atom; each L in the formula is same or different, and may link to one another to form bonds or a ring; the halogen atom is F, Cl, Br or I; the group containing oxygen atom is an alkoxy —OR²³, tetrahydrofuran, or diethyl ether; the group containing nitrogen atom is —NR⁴
R²³, R²⁴, R²⁵, R²⁶, R²⁷, respectively, is H, hydrocarbyl group of C₁-C₃₀ or aryl group of C₅-C₅₀, and these groups are same or different, and R²⁴ with R²⁵, R²⁶ with R²⁷ may form a bond or to a ring;
M is a Group IV to Group VI transition metal.

7. The method for preparing a single-site Ziegler-Natta catalyst as recited in claim 6, wherein the metal complex is a titanium(IV) compound, zirconium (IV) compound, chromium (III) compound, or vanadium compound;

8. The method for preparing a single-site Ziegler-Natta catalyst as recited in claim 7, wherein the Titanium compound is TiCl₄, TiCl₄(THF)₂, Ti(OCH₃)Cl₃, or Ti(OC₂H₅)Cl₃, and THF being tetrahydrofuran.

9. The method for preparing a single-site Ziegler-Natta catalyst as recited in claim 6, wherein the metal complex is TiCl₄, TiCl₄(THF)₂, Ti(NMe₂)₄, Ti(CH₂Ph)₄, ZrCl₄, Zr(NMe₂)₄, CrCl₃, CrCl₃(THF)₃, VCl₃, or VCl₃(THF)₃;

10. The method for preparing a single-site Ziegler-Natta catalyst as recited in claim 6, wherein the supporter is SiO₂.

11. The method for preparing a single-site Ziegler-Natta catalyst as recited in claim 6, wherein the organic compound containing heteroatom is used as an electronic donor to prepare the single site Ziegler-Natta catalyst in preparation steps of
(1) treating an organic or inorganic solid or a mixture thereof at 30-1000° C. for 1-24 hrs under an inert or reduced atmosphere;
(2) at room temperature to 70° C., dissolving a magnesium compound in an THF to form a solution with a ratio of magnesium compound and THF at 1 g:10-100 mL;
(3) adding to the solution (2) the supporter obtained in (1), a metal complex, and an electron donor sequentially, to form a reaction mixture, and keeping the reaction mixture at room temperature to 100° C. for 2-48 hrs, while weight ratio of the magnesium compound and the supporter is 1:0.1-20; molar ratio of the magnesium compound and the metal complex is 0.5-100:1; molar ratio of the organic compound containing heteroatom and the metal complex is (0.01-10):1.

12. The method for preparing a single-site Ziegler-Natta catalyst as recited in claim 11, wherein the magnesium compound reacts with the metal complex for 2-48 hrs at room temperature to 100° C. before being treated with a pretreated carrier, then, a solid obtained therefrom is treated with the electronic donor for 2-48 hrs at room temperature to 100° C. to provide the Ziegler-Natta catalyst.

13. The method for preparing a single-site Ziegler-Natta catalyst as recited in claim 11, wherein the magnesium compound is treated with a carrier for 2-48 h at room temperature to 100° C. to get a composite carrier, and then react with a solution of the electron donor and the metal complex for 2-48 hrs at room temperature to 100° C. to provide the Ziegler-Natta catalyst.

14. The single site Ziegler-Natta catalyst as prepared by the method as recited in claim 6, wherein the catalyst is suitable for ethylene polymerization, ethylene/α-olefin copolymerization, and ethylene/cycloolefin copolymerization.

15. The single site Ziegler-Natta catalyst as recited in claim 14, wherein an alkyl aluminum, alkyl aluminoxane, or a mixture of two or more is used as a cocatalyst in the polymerization, the cocatalyst being AlEt₃, Al(i-Bu)₃, AlEt₂Cl, Al(n-Hex)₃, MAO, EAO, MMAO, or a mixture of two or more; molar ratio of Al/Ti is 20-1000; α-olefins are the olefins of C₃-C₂₀; cycloolefins are cyclopetene, cyclohexene, norbornene, or their derivatives, and all the α-olefins and cycloolefins optionally contain hydroxyl group, carboxyl group, ester group, or amine group.

16. The single site Ziegler-Natta catalyst as recited in claim 14, wherein the polymerization is run in a slurry process or gas process.

17. The single site Ziegler-Natta catalyst as recited in claim 16, wherein in the slurry polymerization process, the polymerization is generally conducted under 0.1-10 MPa of total pressure with hydrogen pressure being 0-1.0 MPa, temperature being 80-120° C.; the polymerization is conducted under supercritical state or subcritical state in propane, isobutene or hexane.

18. The single site Ziegler-Natta catalyst as recited in claim 16, wherein in the gas polymerization process, the polymerization is conducted under 1.0-10.0 MPa at 40-100° C. in gas fluidized bed or gas autoclave.
